(19) United States
(12) Patent Application Publication
(10) Pub. No.: US 2019/0076098 A1
(43) Pub. Date: Mar. 14, 2019

(54) Artificial Neural Network Based Sleep Disordered Breathing Screening Tool

(71) Applicant: ARIZONA BOARD OF REGENTS ON BEHALF OF THE UNIVERSITY OF ARIZONA, Tucson, AZ (US)

(72) Inventors: Ao Li, Tucson, AZ (US); Stuart F Quan, Tucson, AZ (US); Janet Meiling Roveda, Tucson, AZ (US)

(73) Assignee: ARIZONA BOARD OF REGENTS ON BEHALF OF THE UNIVERSTY OF ARIZONA, Tucson, AZ (US)

(21) Appl. No.: 16/125,177

(22) Filed: Sep. 7, 2018

Related U.S. Application Data
(60) Provisional application No. 62/555,968, filed on Sep. 8, 2017.

(51) Int. Cl. A61B 5/00; A61B 5/1455; G16H 50/30; G06N 3/08; A61B 5/18; A61B 5/107; A61B 5/0205

(52) U.S. Cl. CPC A61B 5/7267; A61B 5/14552; A61B 5/4818; A61B 5/7278; A61B 5/7282; G16H 50/30; A61B 5/021; A61B 5/7203; A61B 5/725; A61B 5/18; A61B 5/1072; A61B 5/0205; A61B 5/4809; G06N 3/08

(57) ABSTRACT

The present disclosure provides systems and methods for determining the presence and severity of sleep disordered breathing in a patient based on the output of a low-cost at-home diagnostic and the results of a health questionnaire. The low-cost at-home diagnostic is a simple photoplethysmographic survey to detect oxygen saturation overnight. Minimum oxygen saturation and other metrics are determined from the photoplethysmographic survey and applied, in combination with the health questionnaire data, to a set of artificial neural networks. Each artificial neural network corresponds to a respective degree of severity of sleep disordered breathing, according to rate of occurrence of apnea and hypopnea events during sleep. Each artificial neural network is trained with a respective subset of clinical data generated from a large population of individuals, to reduce both the false positive and false negative rate of the classifier.

### CROSS-REFERENCE TO RELATED APPLICATION

This application claims priority to U.S. Provisional Patent Application No. 62/555,968, filed Sep. 8, 2017, which is incorporated herein by reference.

### BACKGROUND

Sleep disordered breathing (SDB) is a condition in which a person's breathing may diminish, stop, or be otherwise irregular during sleep. This can result in the person exhibiting dangerously decreased blood oxygen saturation levels at a pathological rate during sleep (e.g., higher than a threshold rate per hour). Such decreased oxygen saturation events can be related to periods of apnea and/or hypopnea. Sleep disordered breathing can be related to a variety of health risks, including fatigue, depression, hypertension, heart attack, heart failure, stroke, metabolic disorders, and other serious health conditions.

Accurate diagnosis of the presence and/or severity of SDB can be complicated by the need to perform a full clinical or in-home sleep study. Such diagnostic interventions can be costly and intrusive. Less invasive and/or loss costly diagnostics suffer from diminished sensitivity and accuracy, often requiring more expensive follow-up and verification.

### SUMMARY

An aspect of the present disclosure relates to a method for measuring a degree of severity of sleep disordered breathing of a person, the method including: (i) obtaining a photoplethysmographic signal that is related to a blood oxygenation saturation of the person during a period of time; (ii) determining, based on the photoplethysmographic signal, at least one metric descriptive of the blood oxygenation saturation during the period of time; (iii) receiving an indication of at least one health-related status of the person; (iv) determining a set of classifier outputs corresponding to respective different degrees of severity; and (v) determining a degree of severity of sleep disordered breathing of the person based on the determined set of classifier outputs. Each classifier output is determined using a respective different artificial neural network based on a corresponding set of inputs, where each set of inputs includes (a) one or more of the determined at least one metrics descriptive of the blood oxygenation saturation during the period of time and (b) one or more of the received at least one health-related statuses of the person.

Another aspect of the present disclosure relates to a non-transitory computer-readable medium configured to store at least computer-readable instructions that, when executed by one or more processors of a computing device, cause the computing device to perform computer operations that include: (i) obtaining a photoplethysmographic signal that is related to a blood oxygenation saturation of the person during a period of time; (ii) determining, based on the photoplethysmographic signal, at least one metric descriptive of the blood oxygenation saturation during the period of time; (iii) receiving an indication of at least one health-related status of the person; (iv) determining a set of classifier outputs corresponding to respective different degrees of severity; and (v) determining a degree of severity of sleep disordered breathing of the person based on the determined set of classifier outputs. Each classifier output is determined using a respective different artificial neural network based on a corresponding set of inputs, where each set of inputs includes (a) one or more of the determined at least one metrics descriptive of the blood oxygenation saturation during the period of time and (b) one or more of the received at least one health-related statuses of the person.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE FIGURES

FIG. 1 depicts a flowchart of an example process.

FIG. 2 depicts an example set of artificial neural networks.

FIG. 3A depicts an example photoplethysmographic signal that contains artifacts.

FIG. 3b depicts the photoplethysmographic signal of FIG. 3A after removal of the artifacts.

FIG. 4 depicts an example user interface.

FIG. 5 depicts a flowchart of an example method.

### DETAILED DESCRIPTION

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

### I. Overview

The present disclosure provides a sensitive, accurate diagnostic for detecting the presence and severity of SDB. This includes measuring the blood oxygen saturation of a person during one or more nights' sleep using simple, low-cost diagnostic equipment (e.g., wearable pulse oximeters). The detected blood oxygen saturation data is then processed to generate a number of metrics that are descriptive of the blood oxygenation saturation during the one or more nights' sleep. Such metrics can include a minimum blood oxygen saturation, a percent of time that the blood oxygen saturation was below one or more thresholds, or other metrics. These metrics are then combined with information about one or more health-related statuses of the person and applied to a set of artificial neural network classifiers. Each classifier corresponds to a respective degree of severity of SDB. The outputs of the classifiers can be used to determine whether the person exhibits SDB (e.g., based on whether any of the classifier outputs exceed a threshold) and/or to determine the severity of SDB exhibited by the person (e.g., based on the severity of the highest-severity classifier whose output exceeds a threshold).

The benefits of such a method, relative to a full sleep study or other clinical assessment, is that the classifiers can be trained to accept data that is relatively easy to access while rendering a predicted level of severity of SDB that is accurate. For example, the input data can include demographic information about a person (e.g., age, BMI, neck circumference, dietary habits) as well as one or more metrics determined from an overnight photoplethysmograph, which can be measured using a low-cost, non-prescription device. Additionally, these data can be generated by the person on their own initiative, at low cost and without scheduling an appointment with a clinician. For example, the methods described herein could be implemented as part of a cellphone app, or via a website or other web-based interface. Accordingly, the methods described herein facilitate the detection and quantification of Sleep Disordered Breathing in an accurate, low-cost, and accessible manner. Thus, these methods may increase access to care, reduce cost of care, and promote the early detection and treatment of SDB, improving quality of life and reducing the pain and cost of the negative health effects that can result from SDB.

Health-related statuses of the person can include demographic, anatomical, historical, or other data about the health and behavior of the person. For example, a health related status of the person could include an age, a weight, a height, a sex, a body mass index, a blood pressure, a cholesterol level, a red blood cell count, a resting heart rate, a neck circumference, a body fat percent, a basal metabolic rate, a diabetes diagnosis or related drug dosing, a history or stroke, heart disease, heart failure, or other medical history, or some other information about the anatomy or physiology of the person's body. Additionally or alternatively, a health related status of the person could include a frequency the person snores, a person's dietary patterns, a person's daily caloric intake, a frequency of exercise, a frequency at which the person falls asleep while driving, a frequency at which the person falls asleep while sitting in public, a frequency at which the person falls asleep while sitting and talking, or some other information about the person's conscious or unconscious behavior or habits.

### II. Example Measurement of the Degree of Sleep Disordered Breathing

Previously, it was necessary to undergo extensive clinical interventions in order to determine the presence and severity of sleep disordered breathing (SDB) in a person. This determination could include an initial clinical consult, followed by one or more sleep studies wherein the person is instrumented and observed overnight, oftentimes in a hospital or outpatient facility. The data generated by these clinical assessments was then analyzed by a clinician to diagnose the presence of SDB and/or to determine the severity of SDB in the person. This process involves a great deal of expensive clinician time, expensive and difficult-to-operate equipment, and/or space in a hospital and/or outpatient clinic. Accordingly, this process can be expensive, requires significant time and effort on the part of patients, and may require waiting in a waiting list for local clinical resources to become available.

The systems and methods described herein allow SDB to be detected and quantified in an individual using inexpensive, over-the-counter hardware in the individual's own home. These methods can reduce the cost of obtaining such a determination, increase the ease of access to such a determination (by allowing the individual to pursue the determination in their own time, according to their own schedule without involving the schedule of any clinicians), and reduce crowding and unnecessary use of limited clinical resources (by allowing those without SDB, or with clinically irrelevant levels of SDB, to avoid undergoing a full clinical assessment).

The systems and methods described herein are able to provide these benefits by employing an array of artificial neural networks (ANNs) or other machine learning classifiers that have been trained on an expansive training set consisting of physiological, demographic, and other health measures that can be easily generated by an individual in their own home, by providing the information (e.g., as part of a self-history survey) and/or by using low-cost instrumentation that can be operated by a layperson. For example, the physiological information provided to the classifiers (e.g., ANNs) can include an overnight minimum oxygen saturation, an amount of sleep time spent below a specified threshold oxygen saturation, a pulse rate variability, or some other measures that can be determined from photoplethysmographic data. Such photoplesthysmographic data can be generated by the individual by applying a finger-mounted or otherwise configured commercial photoplethysmographic (PPG) pulse oximeter device to themselves overnight, as they sleep.

The array of classifiers (e.g., ANNs) is trained such that each classifier predicts a respective minimum degree of SDB. For example, a first ANN is trained to provide a “true” output when presented with the physiological and health status information for an individual exhibiting a combined rate of occurrence of apnea and hypopnea that is greater than five times per hour, while second, third, fourth, fifth, and sixth ANNs are trained to provide a “true” output when presented with the information for an individual exhibiting a combined rate of occurrence of apnea and hypopnea that is greater than ten, fifteen, twenty, twenty five, and thirty times per hour, respectively. The set of outputs of the ANNs can then be used to measure a degree of severity of SDB likely to be exhibited by the individual. For example, the level of SDB could be determined according to the lowest SDB level that corresponds to an ANN that generated a “true” output for an individual (e.g., for outputs of “≥5:false,” “≥10:true,” “≥15:false,” “≥20:true,” “≥25:true,” “≥30:true,” the determined level of SDB could be “≥10”). In another example, the level of SDB could be determined according to the lowest SDB level that corresponds to an ANN that generated a “true” output for an individual but for which no lower-level ANN generated a “true” output (e.g., for outputs of “≥5:false,” “≥10:true,” “≥15:false,” “≥20:true,” “≥25:true,” “≥30:true,” the determined level of SDB could be “≥20”). Other methods of generating a level of SDB for an individual, based on an array of ANN outputs for the individual, are anticipated (e.g., providing the ANN outputs to another ANN or other classifier that has been trained to perform such a determination).

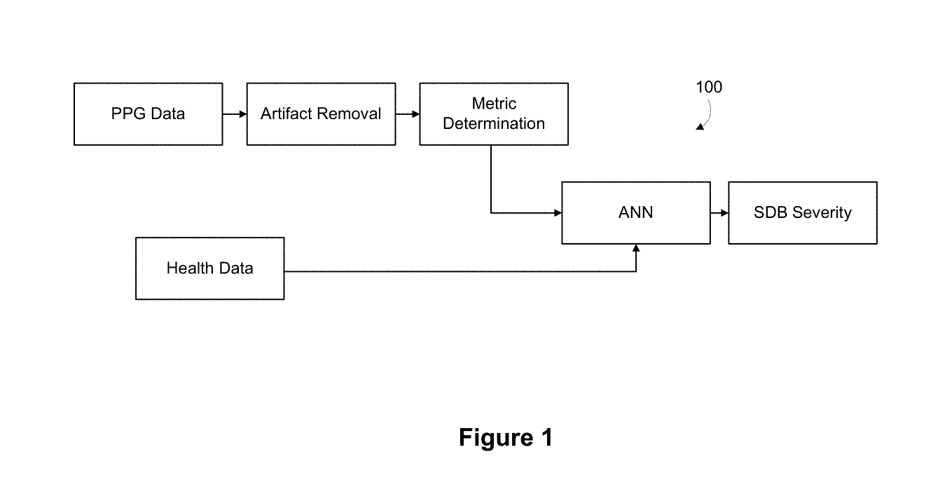

FIG. 1 is a flowchart of a method as described herein. The method 100 includes obtaining a photoplethysmographic signal (“PPG Data”) for a person that represents a blood oxygen saturation of the person during a period of time (e.g., an overnight period of time, while the person sleeps). Optionally, artifacts that may be present in the photoplethysmographic signal may be removed (“Artifact Removal”), e.g., by filtering, detection and removal of discrete artifacts from the signal, or some other method. At least one metric descriptive of the photoplethysmographic signal is then determined (“Metric Determination”) from the photoplethysmographic signal and/or from the artifact-free or otherwise filtered photoplethysmographic signal. The method 100 also includes obtaining at least one health-related status of the person (“Health Data”), e.g., from a website interface, from an app on the person's phone, from a database that contains such information for the person, of via some other source. The at least one metric descriptive of the photoplethysmographic signal and the at least one health-related status of the person are then provided to an array of ANNs (or other machine learning classifiers) and the respective outputs of the ANNs are determined therefrom (“ANN”). These outputs are then used, as described elsewhere herein, to determine an SDB severity for the person (“SBD Severity”).

The at least one metric descriptive of the photoplethysmographic signal could include a variety of statistics or other information determined from a photoplethysmographic signal. For example, the at least one metric could include one or more of a minimum blood oxygenation saturation during the period of time, a percent of the period of time during which the blood oxygenation saturation is below a specified level (e.g., 70%, 75%, 80%, 85%, 90%, or 95%), a maximum pulse rate, a minimum pulse rate, a difference between a maximum pulse rate and a minimum pulse rate, a level of variability of pulse rate, a number or rate of discrete instances of increased pulse rate variability, or some other metrics.

The at least one health-related status of the person could include a variety of demographic, personal history, dietary, medical history, lifestyle, or other information for the person that could be based on self-reporting (e.g., via a survey or other instrument provided, e.g., via a user interface of a cellphone or other computing device), access to one or more databases (e.g., a database containing a record of the person's exercise or other history), or some other information source. For example, the at least one health-related status of the person could include an age, a body mass index, a neck circumference, a frequency of snoring, a frequency of falling asleep while driving, a frequency of falling asleep while inactive in a public place, a frequency of falling asleep while sitting and talking, sex, diastolic blood pressure, systolic blood pressure, a history of (e.g., having experienced at least once) heart attack, heart failure, stroke, hypertension, or diabetes, or some other health status.

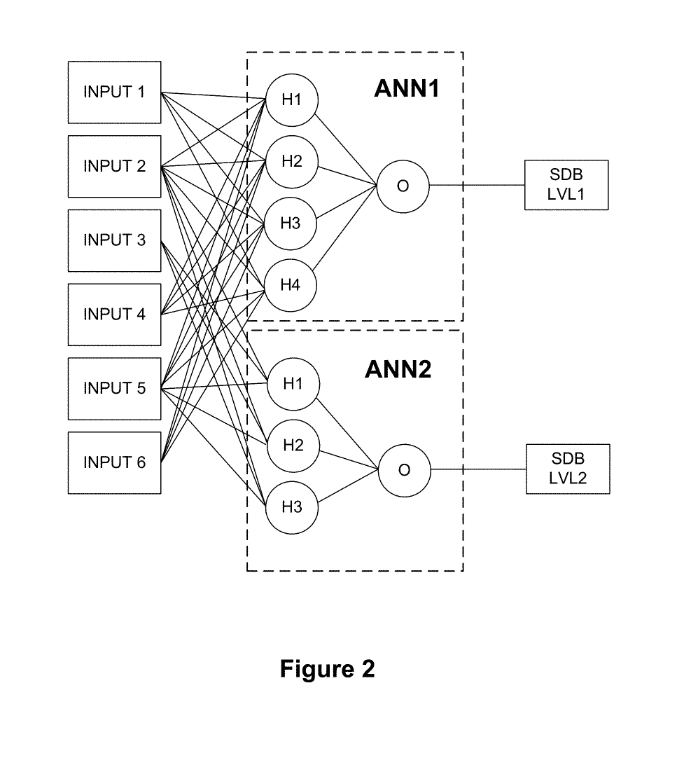

Each of the ANNs (or other machine learning classifiers) used to determine the level of SDB of a person could have a structure, and have been trained, to predict a respective level of SDB of the person. For example, the ANNs could include a multi-layer perceptron or some other ANN structure(s) trained to receive at least one health-related status of the person and at least one metric descriptive of a photoplethysmographic signal measured from the person. FIG. 2 illustrates, by way of example, two ANNs (“ANN1” and “ANN2”) of a predictive system as described herein. Each of the ANNs receives inputs from a set of inputs (“INPUT1” -“INPUT 6”) into a respective set of hidden layer neurons (“H1” through “H4” for ANN1 and “H1” through “H3” for ANN2). Outputs of the hidden layer neurons are directed to respective output layer neurons (“0”) which generate the respective outputs of the ANNs (“SDB LVL1” for ANN1 and “SDB LVL2” for ANN2).

Each of the ANNs of a predictive system described herein (e.g., ANN1 and ANN2) could have a respective different configuration, e.g., a respective number of hidden layer units (e.g., four and three, respectively, for ANN1 and ANN2), a respective subset of inputs received from a larger set of possible inputs (e.g., inputs 1, 2, 4, 5, and 6 for ANN1 and 2, 3, and 5 for ANN2), a respective hidden layer and/or output layer activation function (e.g., logistic, hyperbolic tangent, etc.), or some other configuration parameters.

In an example, a set of six ANNs were trained to predict whether a person would exhibit a combined rate of occurrence of apnea and hypopnea that is greater than five, ten, fifteen, twenty, twenty five, and thirty times per hour, respectively. These neural networks were trained based on a set of training data that included records corresponding to a plurality of persons and that included, for each person, information about at least one metric descriptive of blood oxygen saturation during a clinical assessment and at least one health-related status. A grid search method was used to determine the best set of inputs, from the at least one metric descriptive of blood oxygen saturation and the at least one health-related status, for each of the ANNs. The ANNs were trained using backpropagation and a limited memory Broyden-Fletcher-Goldfarb-Shanno algorithm to optimize each ANN relative to the receiver operating characteristic of the ANNs for detecting their respective outputs (i.e., their respective predicted levels of SDB). Other parameters of the ANNs, including the number of hidden layers and the output function for neurons of the hidden layers, were also optimized based on the training data. The output layers of the ANNs used a logistic output function (though hyperbolic tangent or other output functions are also possible). The results of this training were as follows.

For the ANN trained to generate an output corresponding to the person exhibiting a combined rate of occurrence of apnea and hypopnea that is greater than five times per hour, the ANN had four hidden layer neurons that used logistic activation functions and received as inputs age, body mass index (BMI), neck circumference, minimum blood oxygenation saturation during the period of time, percents of the period of time during which the blood oxygenation saturation was below 90% and below 95%, and frequency of snoring.

For the ANN trained to generate an output corresponding to the person exhibiting a combined rate of occurrence of apnea and hypopnea that is greater than ten times per hour, the ANN had three hidden layer neurons that used hyperbolic tangent activation functions and received as inputs age, BMI, neck circumference, minimum blood oxygenation saturation during the period of time, percents of the period of time during which the blood oxygenation saturation is below 90%, 95%, and 85%, and a frequency of snoring.

For the ANN trained to generate an output corresponding to the person exhibiting a combined rate of occurrence of apnea and hypopnea that is greater than fifteen times per hour, the ANN had six hidden layer neurons that used logistic activation functions and received as inputs age, BMI, neck circumference, minimum blood oxygenation saturation during the period of time, percents of the period of time during which the blood oxygenation saturation is below 90%, 95%, and 85%, and frequency of snoring.

For the ANN trained to generate an output corresponding to the person exhibiting a combined rate of occurrence of apnea and hypopnea that is greater than twenty times per hour, the ANN had four hidden layer neurons that used logistic activation functions and received as inputs age, BMI, neck circumference, minimum blood oxygenation saturation during the period of time, percents of the period of time during which the blood oxygenation saturation is below 90%, 95%, and 85%, and frequency of snoring.

For the ANN trained to generate an output corresponding to the person exhibiting a combined rate of occurrence of apnea and hypopnea that is greater than twenty-five times per hour, the ANN had ten hidden layer neurons that used hyperbolic tangent activation functions and received as inputs age, BMI, neck circumference, frequency of snoring, minimum blood oxygenation saturation during the period of time, percents of the period of time during which the blood oxygenation saturation is below 90%, 95%, 85%, 80%, and 75%, and frequency of falling asleep while inactive in a public place.

For the ANN trained to generate an output corresponding to the person exhibiting a combined rate of occurrence of apnea and hypopnea that is greater than thirty times per hour, the ANN had seven hidden layer neurons that used hyperbolic tangent activation functions and received as inputs age, BMI, neck circumference, minimum blood oxygenation saturation during the period of time, percents of the period of time during which the blood oxygenation saturation is below 90%, 95%, 85%, and 80%, and frequency of snoring.

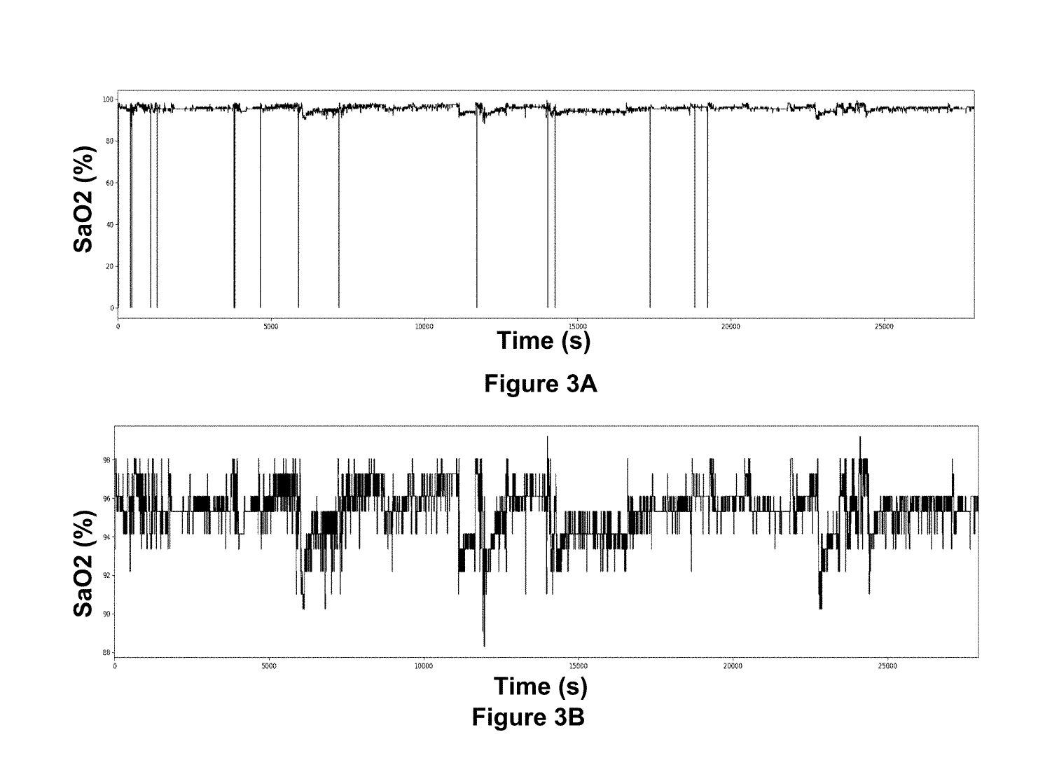

The measured photoplethysmographic signal can include artifacts (due, e.g., to relative movement between the person being measured and a pulse oximeter or other photoplethysmographic sensor being used to generate the photoplethysmographic signal). In order to improve the quality (e.g., accuracy) of the at least one metric descriptive of blood oxygen saturation measured from the person, the photoplethysmographic signal can be filtered or otherwise processed in order to remove these artifacts. An example of such an artifact-containing photoplethysmographic signal is illustrated in FIG. 3A. The artifacts are represented by the downward spikes in the photoplethysmographic signal, while the remainder of the photoplethysmographic signal represents the actual blood oxygen saturation level.

Such artifacts could be removed in a variety of ways. In some examples, a lowpass filter could be used to filter the photoplethysmographic signal. In another example, samples of the photoplethysmographic signal that are outside of a specified range of values (e.g., less than a minimum value) could be discarded. In yet another level, an ANN could be trained to identify artifacts in a photoplethysmographic signal and the ANN-identified artifacts could be removed from the photoplethysmographic signal. Such an ANN could be trained based on samples of photoplethysmographic signals that have been annotated to indicate artifacts present in the photoplethysmographic signals.

Such an ANN could be a long short term memory recurrent neural network. FIG. 3B illustrates the results of using such a neural network to identify and remove artifacts from a photoplethysmographic signal. The photoplethysmographic signal of FIG. 3B is photoplethysmographic signal of 3A, with the artifacts that have identified using the long short term memory recurrent neural network removed. This method can also can be used to identify artifacts within other biomedical signals (e.g. electroencephalogram, electromyography, electrocardiogram) that could be used, according to the methods described herein, to provide physiological data similarly to a photoplethysmographic signal.

A determined degree of severity of sleep disordered breathing of a person, determined according to the methods disclosed herein, could be used to inform the treatment of the person for SDB and/or for one or more other conditions related to SDB. For example, upon determining that the person is experiencing mild SDB (e.g., determining the person has a degree of severity of SDB greater than a “mild” threshold level), the person could be instructed to sleep on their side, to improve their sleep hygiene (e.g., to refrain from watching TV in bed, to maintain a regular sleep schedule), to use nasal strips, to use a continuous positive airway pressure (CPAP) machine, to use an oral appliance, to lose weight or exercise more, or to take some other measure. Upon determining that the person is experiencing moderate to severe SDB (e.g., determining the person has a degree of severity of SDB greater than a “moderate” and/or “severe” threshold level), additional or alternative interventions to those listed above could be pursued, e.g., a greater emphasis could be applied to the use of CPAP or oral appliances, surgical interventions could be pursued (e.g., bariatric surgery, the removal tonsils and/or adenoids, uvulopalatopharyngoplasty, radiofrequency ablation of occlusive tissues, jaw repositioning, the installation of one or more implants to maintain the airway, implanting a hypoglossal nerve stimulator), and/or some other intervention. The therapeutic intervention could be directed to the treatment of sleep apnea, insomnia or some other condition related to SDB (e.g., obesity).

In examples wherein the determined level of SDB rules out the presence of SDB and/or rules out the likelihood that SDB is the cause of a symptom of interest (e.g., due to no SDB being detected and/or the detected level of SDB being below a threshold level), other disorders or diseases could be investigated. For example, if no SDB was detected and/or the detected level of SDB was very low, other causes of a sleep problem such as idiopathic hypersomnia, narcolepsy, restless legs syndrome, periodic limb movement disorder or behaviorally induced insufficient sleep can be investigated.

### III. Example User Interface

The photoplethysmographic signal and health related status information used by the systems and methods described herein could be accessed in a variety of ways, and the methods performed on that data by a variety of systems. In some examples, a cellphone or other computing device (e.g., a personal computer) operated by a person could include programming (e.g., a cellphone app) configured to obtain the data and to apply the data to generate a level of SDB for the person. For example, the programming could access the photoplethysmographic signal data from a pulse oximeter (e.g., via a wired or wireless communication channel) and could obtain at least one health related status by presenting, to the person, a user interface requesting health-related information from the person (e.g., in the form of a survey or questionnaire). Additionally or alternatively, a server, cloud computing service, or other computational system that is remote from the person could access the relevant data about the person (e.g., by receiving an upload of the photoplethysmographic signal data from the person via an app or other method, by presenting a survey or questionnaire to the person in the form of a website or web portal in order to obtain the health-related information). The remote computing system could then provide the determined SDB level to the person and/or to their physician (e.g., via a website, via an email, via a phonecall or text message).

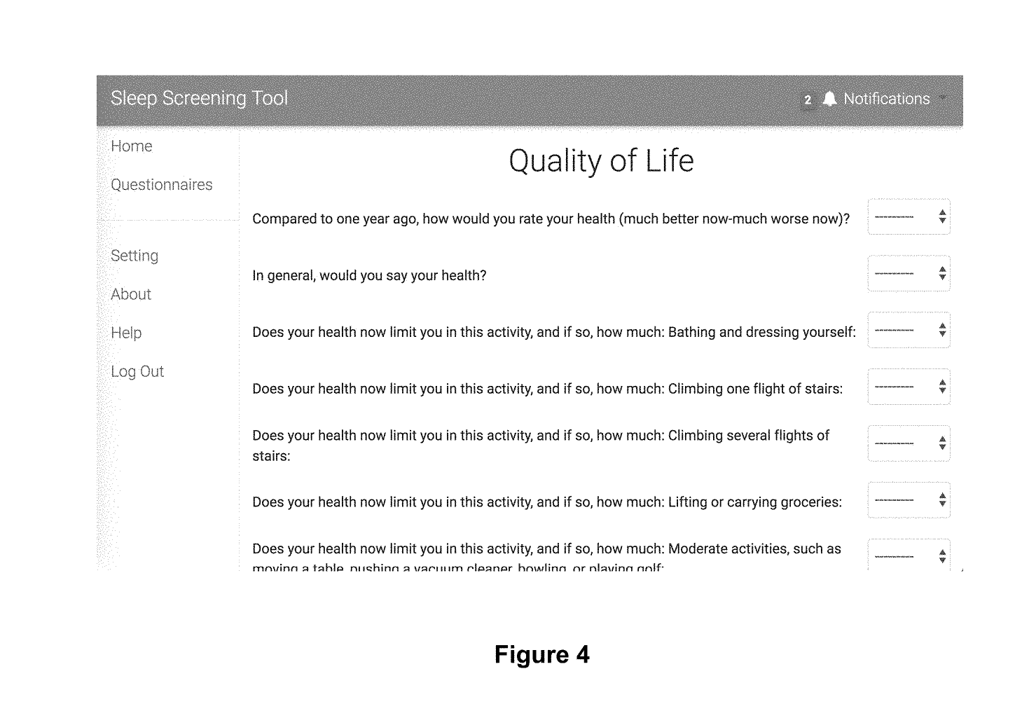

FIG. 4 illustrates an example user interface of a cellphone, tablet, personal computer, or some other computing system. The user interface could be provided as part of an app or other programming present on the device. Additionally or alternatively, the user interface could be provided as a website. The user interface includes a questionnaire via which a person could provide information about a number of health-related statuses of the person. The example user interface of FIG. 4 includes question about a person's self-reported current and past levels of overall health and whether the person is limited in a variety of activities by their health.

Such questionnaire could be accessed from a “home screen” or other user interface function (e.g., website) that permits access to other functions. For example, a function to permit uploading of photoplethysmographic signal data (e.g., from a pulse oximeter) via a wired (e.g., USB) or wireless (e.g., WiFi, Bluetooth) connection. In examples wherein the user interface is provided via a website, the upload function could transmit such photoplethysmographic signal data to a remote system (e.g., a cloud computing system) that could then use the uploaded photoplethysmographic signal data, in combination with health related status information for a person, to predict a level of SDB exhibited by the person. Another function could include contacting a physician's office to schedule a follow-up visit (e.g., if the predicted SDB level is greater than a threshold level), accessing the person's personal calendar to facilitate such scheduling, providing a list of specialists who treat SDB or related health issues, providing links to information about SDB, or other resources.

### IV. Example Methods

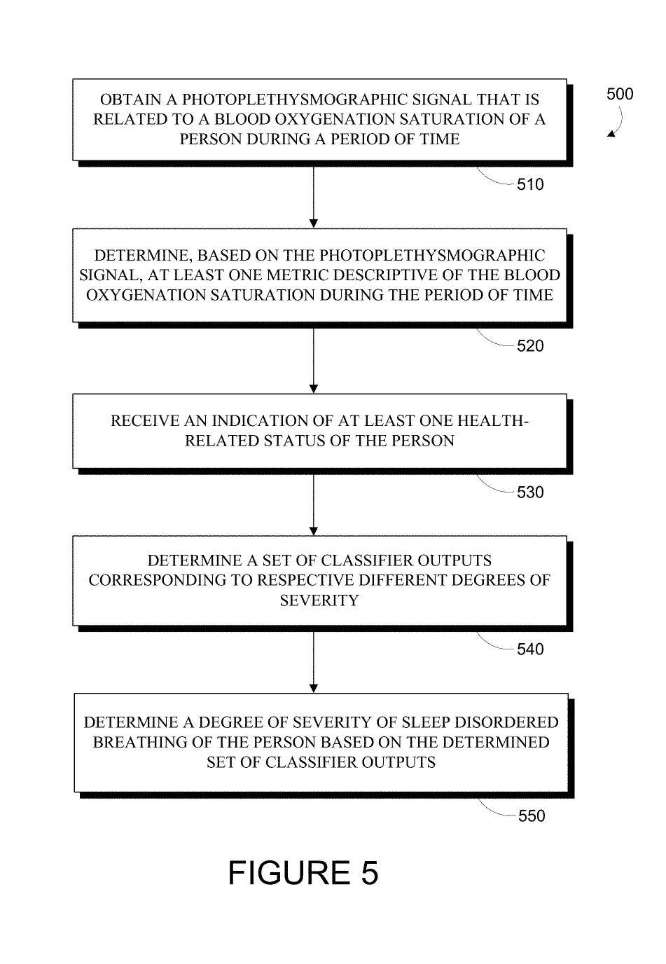

FIG. 5 is a flowchart of a method 500 for measuring a degree of severity of sleep disordered breathing of a person. The method 500 includes obtaining a photoplethysmographic signal that is related to a blood oxygenation saturation of a person during a period of time (510). This can include operating a wearable or otherwise portable photoplethysmographic sensor (e.g., a finger-mounted pulse oximeter) to generate the photoplethysmographic signal during the period of time, e.g., overnight. The photoplethysmographic signal generated thereby could then be obtained by a processor or other computing system or device performing the method, e.g., via a wired or wireless connection. For example, the person could upload the photoplethysmographic signal from the photoplethysmographic sensor to their cellphone (e.g., via a Bluetooth wireless link), which could run an app performing the method 500. Additionally or alternatively, the photoplethysmographic signal could be uploaded by the person to a cloud computing system (e.g., via an app installed on their cellphone, which receives the photoplethysmographic signal from the photoplethysmographic sensor via a Bluetooth wireless link).

The method 500 additionally includes determining, based on the photoplethysmographic signal, at least one metric descriptive of the blood oxygenation saturation during the period of time (520). The method 500 also includes receiving an indication of at least one health-related status of the person (530). This could include receiving the indication of at least one health-related status of the person via a user interface of a cellphone, computer, or other device. For example, the indication could be received via a user interface of an app running on a cellphone or a user interface of a cellphone or other computing device providing access to a website (e.g., an app or a website providing an interface for the person to input demographic or other health data). Additionally or alternatively, the indication of at least one health-related status of the person could be obtained from a database containing such information for the person (e.g., a database maintained by the person's physician).

The method 500 additionally includes determine a set of classifier outputs corresponding to respective different degrees of severity (540). Each classifier output is determined using a respective different artificial neural network or other classifier structure based on a corresponding set of inputs, with each set of inputs including (i) one or more of the determined at least one metrics descriptive of the blood oxygenation saturation during the period of time and (ii) one or more of the received at least one health-related statuses of the person. The set of inputs applied to each of the classifiers could be the same, or could differ according to the classifier.

The method 500 additionally includes determining a degree of severity of sleep disordered breathing of the person based on the determined set of classifier outputs (550). This could include determining the severity of SDB according to the lowest level of SDB that corresponds to an ANN that output a “true” value. For example, if a set of ANNs corresponds to respectively higher levels of SDB, according to integer values one through six, and the ANNs corresponding to values “2,” “4,” “5,” and “6” output “true” values, the determined severity of SDB determined therefrom could be “2.”In another example, the determined severity of SDB could correspond to the lowest level of SDB that corresponds to an ANN that output a “true” value but for which no ANN corresponding to a higher SDB value output a “true” value. For example, if a set of ANNs corresponds to respectively higher levels of SDB, according to integer values one through six, and the ANNs corresponding to values “2,” “4,” “5,” and “6” output “true” values, the determined severity of SDB determined therefrom could be “4.” Other methods of determining a severity of SDB from the outputs generated from an array of ANNs (or other machine learning classifiers) as described herein are anticipated.

### V. Conclusion

The above detailed description describes various features and functions of the disclosed systems, devices, and methods with reference to the accompanying figures. In the figures, similar symbols typically identify similar components, unless context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

We claim:

1. A method for measuring a degree of severity of sleep disordered breathing of a person, comprising:
obtaining a photoplethysmographic signal, wherein the photoplethysmographic signal is related to a blood oxygenation saturation of the person during a period of time;
determining, based on the photoplethysmographic signal, at least one metric descriptive of the blood oxygenation saturation during the period of time;
receiving an indication of at least one health-related status of the person;
determining a set of classifier outputs corresponding to respective different degrees of severity, wherein each classifier output is determined using a respective different artificial neural network based on a corresponding set of inputs, wherein each set of inputs comprises (i) one or more of the determined at least one metrics descriptive of the blood oxygenation saturation during the period of time and (ii) one or more of the received at least one health-related statuses of the person; and
determining a degree of severity of sleep disordered breathing of the person based on the determined set of classifier outputs.

2. The method of claim 1, wherein determining, based on the photoplethysmographic signal, at least one metric descriptive of the blood oxygenation saturation during the period of time comprises determining, based on the photoplethysmographic signal, at least one of: (i) a minimum blood oxygenation saturation during the period of time; (ii) a percent of the period of time during which the blood oxygenation saturation is below 70%; (iii) a percent of the period of time during which the blood oxygenation saturation is below 75%; (iv) a percent of the period of time during which the blood oxygenation saturation is below 80%; (v) a percent of the period of time during which the blood oxygenation saturation is below 85%; (vi) a percent of the period of time during which the blood oxygenation saturation is below 90%; or (vii) a percent of the period of time during which the blood oxygenation saturation is below 95%.

3. The method of claim 1, wherein receiving an indication of at least one health-related status of the person comprises receiving an indication of at least one of: (i) an age of the person; (ii) a body mass index of the person; (iii) a neck circumference of the person; (iv) a frequency of snoring exhibited by the person; (v) a frequency of falling asleep while driving exhibited by the person; (vi) a frequency of falling asleep while inactive in a public place exhibited by the person; (vii) a frequency of falling asleep while sitting and talking exhibited by the person; (viii) a sex of the person; (ix) a diastolic blood pressure of the person; or (x) a systolic blood pressure of the person.

4. The method of claim 1, wherein determining a set of classifier outputs corresponding to respective different degrees of severity comprises:
determining a first classifier output corresponding to the person exhibiting a combined rate of occurrence of apnea and hypopnea that is greater than five times per hour;
determining a second classifier output corresponding to the person exhibiting a combined rate of occurrence of apnea and hypopnea that is greater than ten times per hour;
determining a third classifier output corresponding to the person exhibiting a combined rate of occurrence of apnea and hypopnea that is greater than fifteen times per hour;
determining a fourth classifier output corresponding to the person exhibiting a combined rate of occurrence of apnea and hypopnea that is greater than twenty times per hour;
determining a fifth classifier output corresponding to the person exhibiting a combined rate of occurrence of apnea and hypopnea that is greater than twenty-five times per hour; and
determining a sixth classifier output corresponding to the person exhibiting a combined rate of occurrence of apnea and hypopnea that is greater than thirty times per hour.

5. The method of claim 4, wherein:
determining the first classifier comprises using a first artificial neural network based on a first set of inputs, wherein the first set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, and a frequency of snoring exhibited by the person;
determining the second classifier comprises using a second artificial neural network based on a second set of inputs, wherein the second set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the third classifier comprises using a third artificial neural network based on a third set of inputs, wherein the third set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the fourth classifier comprises using a fourth artificial neural network based on a fourth set of inputs, wherein the fourth set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the fifth classifier comprises using a fifth artificial neural network based on a fifth set of inputs, wherein the fifth set of inputs comprises: an age of the person, a body mass index of the person, a neck circumference of the person, a frequency of snoring exhibited by the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, a percent of the period of time during which the blood oxygenation saturation is below 80%, a percent of the period of time during which the blood oxygenation saturation is below 75%, and a frequency of falling asleep while inactive in a public place exhibited by the person; and
determining the sixth classifier comprises using a sixth artificial neural network based on a sixth set of inputs, wherein the sixth set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, a percent of the period of time during which the blood oxygenation saturation is below 80%, and a frequency of snoring exhibited by the person.

6. The method of claim 5, wherein:
the first artificial neural network includes a hidden layer that uses logistic activation functions and an output layer that uses logistic activation functions;
the second artificial neural network includes a hidden layer that uses hyperbolic tangent activation functions and an output layer that uses logistic activation functions;
the third artificial neural network includes a hidden layer that uses logistic activation functions and an output layer that uses logistic activation functions;
the fourth artificial neural network includes a hidden layer that uses logistic activation functions and an output layer that uses logistic activation functions;
the fifth artificial neural network includes a hidden layer that uses hyperbolic tangent activation functions and an output layer that uses logistic activation functions; and
the sixth artificial neural network includes a hidden layer that uses hyperbolic tangent activation functions and an output layer that uses logistic activation functions.

7. The method of claim 1, further comprising:
based on the determining a degree of severity of sleep disordered breathing of the person, providing a therapeutic intervention to the person.

8. The method of claim 1, further comprising:
training the artificial neural networks based on a set of training data, wherein the set of training data comprises records corresponding to a plurality of persons, wherein a record corresponding to a particular person of the plurality of persons comprises information about:
at least one metric descriptive of a blood oxygenation saturation of the particular person during a clinical assessment;
at least one health-related status of the particular person; and
a measured degree of severity of sleep disordered breathing of the particular person.

9. The method of claim 8, wherein training the artificial neural networks comprises using backpropagation and a limited memory Broyden-Fletcher-Goldfarb-Shanno algorithm to optimize the artificial neural networks relative to an area under a receiver operating characteristic curve of the artificial neural networks.

10. The method of claim 8, wherein training the artificial neural networks comprises determining an input set for each of the artificial neural networks, wherein determining an input set for a particular neural network comprises using a random forest method to select (i) one or more of the determined at least one metrics descriptive of the blood oxygenation saturation during the period of time and (ii) one or more of the received at least one health-related statuses of the person.

11. The method of claim 1, wherein receiving an indication of at least one health-related status of the person comprises receiving an indication of the at least one health-related status of the person from a database.

12. The method of claim 1, wherein receiving an indication of at least one health-related status of the person comprises operating a user interface to receive user input indicative of the at least one health-related status of the person.

13. The method of claim 1, wherein obtaining a photoplethysmographic signal comprises operating a pulse oximeter to generate the photoplethysmographic signal.

14. The method of claim 1, further comprising:
applying an artificial neural network to the photoplethysmographic signal to identify artifacts within the photoplethysmographic signal; and
removing the identified artifacts from the photoplethysmographic signal to generate a filtered photoplethysmographic signal, wherein determining, based on the photoplethysmographic signal, at least one metric descriptive of the blood oxygenation saturation during the period of time comprises determining the at least one metric descriptive of the blood oxygenation saturation during the period of time based on the filtered photoplethysmographic signal.

15. A non-transitory computer-readable medium, configured to store at least computer-readable instructions that, when executed by one or more processors of a computing device, cause the computing device to perform computer operations comprising:
obtaining a photoplethysmographic signal, wherein the photoplethysmographic signal is related to a blood oxygenation saturation of a person during a period of time;
determining, based on the photoplethysmographic signal, at least one metric descriptive of the blood oxygenation saturation during the period of time;
receiving an indication of at least one health-related status of the person;
determining a set of classifier outputs corresponding to respective different degrees of severity, wherein each classifier output is determined using a respective different artificial neural network based on a corresponding set of inputs, wherein each set of inputs comprises (i) one or more of the determined at least one metrics descriptive of the blood oxygenation saturation during the period of time and (ii) one or more of the received at least one health-related statuses of the person; and
determining a degree of severity of sleep disordered breathing of the person based on the determined set of classifier outputs.

16. The non-transitory computer-readable medium of claim 15, wherein determining, based on the photoplethysmographic signal, at least one metric descriptive of the blood oxygenation saturation during the period of time comprises determining, based on the photoplethysmographic signal, at least one of: (i) a minimum blood oxygenation saturation during the period of time; (ii) a percent of the period of time during which the blood oxygenation saturation is below 70%; (iii) a percent of the period of time during which the blood oxygenation saturation is below 75%; (iv) a percent of the period of time during which the blood oxygenation saturation is below 80%; (v) a percent of the period of time during which the blood oxygenation saturation is below 85%; (vi) a percent of the period of time during which the blood oxygenation saturation is below 90%; or (vii) a percent of the period of time during which the blood oxygenation saturation is below 95%.

17. The non-transitory computer-readable medium of claim 15, wherein receiving an indication of at least one health-related status of the person comprises receiving an indication of at least one of: (i) an age of the person; (ii) a body mass index of the person; (iii) a neck circumference of the person; (iv) a frequency of snoring exhibited by the person; (v) a frequency of falling asleep while driving exhibited by the person; (vi) a frequency of falling asleep while inactive in a public place exhibited by the person; (vii) a frequency of falling asleep while sitting and talking exhibited by the person; (viii) a sex of the person; (ix) a diastolic blood pressure of the person; or (x) a systolic blood pressure of the person.

18. The non-transitory computer-readable medium of claim 15, wherein determining a set of classifier outputs corresponding to respective different degrees of severity comprises:
determining a first classifier output corresponding to the person exhibiting a combined rate of occurrence of apnea and hypopnea that is greater than five times per hour;
determining a second classifier output corresponding to the person exhibiting a combined rate of occurrence of apnea and hypopnea that is greater than ten times per hour;
determining a third classifier output corresponding to the person exhibiting a combined rate of occurrence of apnea and hypopnea that is greater than fifteen times per hour;
determining a fourth classifier output corresponding to the person exhibiting a combined rate of occurrence of apnea and hypopnea that is greater than twenty times per hour;
determining a fifth classifier output corresponding to the person exhibiting a combined rate of occurrence of apnea and hypopnea that is greater than twenty-five times per hour; and
determining a sixth classifier output corresponding to the person exhibiting a combined rate of occurrence of apnea and hypopnea that is greater than thirty times per hour.

19. The non-transitory computer-readable medium of claim 18, wherein:
determining the first classifier comprises using a first artificial neural network based on a first set of inputs, wherein the first set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, and a frequency of snoring exhibited by the person;
determining the second classifier comprises using a second artificial neural network based on a second set of inputs, wherein the second set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the third classifier comprises using a third artificial neural network based on a third set of inputs, wherein the third set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the fourth classifier comprises using a fourth artificial neural network based on a fourth set of inputs, wherein the fourth set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, and a frequency of snoring exhibited by the person;
determining the fifth classifier comprises using a fifth artificial neural network based on a fifth set of inputs, wherein the fifth set of inputs comprises: an age of the person, a body mass index of the person, a neck circumference of the person, a frequency of snoring exhibited by the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, a percent of the period of time during which the blood oxygenation saturation is below 80%, a percent of the period of time during which the blood oxygenation saturation is below 75%, and a frequency of falling asleep while inactive in a public place exhibited by the person; and
determining the sixth classifier comprises using a sixth artificial neural network based on a sixth set of inputs, wherein the sixth set of inputs comprises: an age of the person, a body mass index of the person, a neck circumference of the person, a minimum blood oxygenation saturation during the period of time, a percent of the period of time during which the blood oxygenation saturation is below 90%, a percent of the period of time during which the blood oxygenation saturation is below 95%, a percent of the period of time during which the blood oxygenation saturation is below 85%, a percent of the period of time during which the blood oxygenation saturation is below 80%, and a frequency of snoring exhibited by the person.

20. The non-transitory computer-readable medium of claim 15, wherein the computer operations further comprise:
applying an artificial neural network to the photoplethysmographic signal to identify artifacts within the photoplethysmographic signal; and
removing the identified artifacts from the photoplethysmographic signal to generate a filtered photoplethysmographic signal, wherein determining, based on the photoplethysmographic signal, at least one metric descriptive of the blood oxygenation saturation during the period of time comprises determining the at least one metric descriptive of the blood oxygenation saturation during the period of time based on the filtered photoplethysmographic signal.
